(19) United States
(12) Patent Application Publication
(10) Pub. No.: US 2003/0219712 A1
(43) Pub. Date: Nov. 27, 2003

(54) Addition of glycolysis inhibitor to a pathogen reduction and storage solution

(76) Inventors: Laura Goodrich, Lakewood, CO (US); Raymond P. Goodrich, Lakewood, CO (US)

Correspondence Address: GAMBRO, INC, PATENT DEPARTMENT, 10810 W COLLINS AVE, LAKEWOOD, CO, 80215, US

(21) Appl. No.: 10/355,681

(22) Filed: Jan. 31, 2003

Related U.S. Application Data
(60) Provisional application No. 60/353,319, filed on Feb. 1, 2002.

(51) Int. Cl. C12N 5/00; C12N 5/02

(52) U.S. Cl. 435/2; 435/374

(57) ABSTRACT

This invention relates to the addition of glycolytic inhibitors to solutions used to pathogen reduce and subsequent storage of blood components such as platelets. More particularly, the invention relates to the addition of 2-deoxy-D-glucose to a platelet pathogen reduction and storage solution.

### CROSS REFERENCE To RELATED APPLICATIONS

This application claims priority to U.S. Provisional patent application No. 60/353,319 filed Feb. 1, 2002.

### BACKGROUND OF THE INVENTION

The breakdown of glucose to provide energy to cells is an important mechanism in cellular metabolism. This mechanism, known as glycolysis, produces ATP (adenosine triphosphate) in the absence of oxygen. The production of ATP is essential for cellular energy metabolism.

In the process of glycolysis, a glucose molecule with six carbon atoms is converted into two molecules of pyruvate, each with three carbon atoms. This conversion involves a sequence of nine enzymatic steps that create phosphate-containing intermediates. The cell hydrolyzes two molecules of ATP to drive the early steps, but produces four molecules of ATP in the later steps.

For most animal cells, glycolysis is merely the first stage in the breakdown of sugar into cellular energy, since the pyruvic acid that is formed at the last step quickly enters the cell's mitochondria to be completely oxidized to CO and H₂O in the citric acid cycle. In the case of organisms which are anaerobic (those that do not use molecular oxygen) and for tissues like skeletal muscle that can function under anaerobic conditions, glycolysis can become a major source of the cell's ATP. This also occurs if the mitochondria of the cell is damaged in some way, thereby preventing the cell from entering the citric acid cycle.

Since ATP is essential to continued cell function, when aerobic metabolism is slowed or prevented by lack of oxygen, anaerobic pathways for producing ATP are stimulated and become critical for maintaining cell viability. Here, instead of being degraded in the mitochondria, the pyruvate molecules stay in the cytosol and can be converted into ethanol and CO₂ (as in yeast) or into lactate (as in muscle).

Lactate accumulation in cells causes an increased concentration of hydrogen ions (a decreased pH). If cells undergoing glycolysis are being stored, such a drop in pH might contribute to a decrease in cell quality during cell storage.

Factors which might cause cells to enter glycolysis and thereby accumulate lactic acid or lactate may be events which occur internally in a body such as strokes or infarctions, or may be caused by external events such as treatment of the cells after removal from a body. One example of an external treatment which might cause cells to accumulate lactate is a procedure to inactivate any pathogens which might be contained in cells to be transfused into a recipient. Currently used methods to sterilize pathogenic contaminants which may be present in blood or blood components may cause damage to the mitochondria of the cells being treated. If this occurs, the cells can only make ATP through the glycolysis pathway, causing a buildup of lactic acid in the cell, and a subsequent drop in pH during storage.

One method used to sterilize blood and blood components requires the use of photosensitizers, compounds which absorb light of a defined wavelength and transfer the absorbed energy to an energy acceptor. For example, European Patent application 196,515 published Oct. 8, 1986, suggests the use of non-endogenous photosensitizers such as porphyrins, psoralens, acridine, toluidines, flavine (acriflavine hydrochloride), phenothiazine derivatives, and dyes such as neutral red and methylene blue, as blood additives. Protoporphyrin, which occurs naturally within the body, can be metabolized to form a photosensitizer; however, its usefulness is limited in that it degrades desired biological activities of proteins. Chlorpromazine is also exemplified as one such photosensitizer; however its usefulness is limited by the fact that it should be removed from any fluid administered to a patient after the decontamination procedure because it has a sedative effect.

Goodrich, R. P., et al. (1997), “The Design and Development of Selective, Photoactivated Drugs for Sterilization of Blood Products,” Drugs of the Future 22:159-171 provides a review of some photosensitizers including psoralens, and some of the issues of importance in choosing photosensitizers for decontamination of blood products. The use of texaphyrins for DNA photocleavage is described in U.S. Pat. Nos. 5,607,924 issued Mar. 4, 1997 and 5,714,328 issued Feb. 3, 1998 to Magda et al. The use of sapphyrins for viral deactivation is described in U.S. Pat. No. 5,041,078 issued Aug. 20, 1991 to Matthews, et al. Inactivation of extracellular enveloped viruses in blood and blood components by Phenthiazin-5-ium dyes plus light is described in U.S. Pat. No. 5,545,516 issued Aug. 13, 1996 to Wagner. The use of porphyrins, hematoporphyrins, and merocyanine dyes as photosensitizing agents for eradicating infectious contaminants such as viruses and protozoa from body tissues such as body fluids is disclosed in U.S. Pat. No. 4,915,683 issued Apr. 10, 1990 and related U.S. Pat. No. 5,304,113 issued Apr. 19, 1994 to Sieber et al. The reactivity of psoralen derivatives with viruses has been studied. See, Hearst and Thiry (1977) Nuc. Acids Res. 4:1339-1347; and Talib and Banerjee (1982) Virology 118:430-438. U.S. Pat. Nos. 4,124,598 and 4,196,281 to Hearst et al. suggest the use of psoralen derivatives to inactivate RNA viruses, but include no discussion of the suitability of such inactivated viruses as vaccines.

The mechanism of action of psoralens is described as involving preferential binding to domains in lipid bilayers, e.g. on enveloped viruses and some virus-infected cells. Photoexcitation of membrane-bound agent molecules leads to the formation of reactive oxygen species such as singlet oxygen which causes lipid peroxidation. A problem with the use of psoralens is that they attack cell membranes of desirable components of fluids to be decontaminated, such as red blood cells, and the singlet oxygen produced during the reaction also attacks desired protein components of fluids being treated.

U.S. Pat. No. 4,727,027 issued Feb. 23, 1988 to Wiesehahn, G. P., et al. discloses the use of furocoumarins including psoralen and derivatives for decontamination of blood and blood products, but teaches that steps must be taken to reduce the availability of dissolved oxygen and other reactive species in order to inhibit denaturation of biologically active proteins. Photoinactivation of viral and bacterial blood contaminants using halogenated coumarins is described in U.S. Pat. No. 5,516,629 issued May 14, 1996 to Park, et al. U.S. Pat. No. 5,587,490 issued Dec. 24, 1996 to Goodrich Jr., R. P., et al. and U.S. Pat. No. 5,418,130 to Platz, et al. disclose the use of substituted psoralens for inactivation of viral and bacterial blood contaminants. The latter patent also teaches the necessity of controlling free radical damage to other blood components. U.S. Pat. No. 5,654,443 issued Aug. 5, 1997 to Wollowitz et al. teaches new psoralen compositions used for photodecontamination of blood.

It is known in the art to use photosensitizers in solutions for photodecontamination of blood. For example, U.S. Pat. No. 5,709,991 to Lin et al. teaches the use of psoralen for photodecontamination of platelet preparations and removal of psoralen afterward, U.S. Pat. No. 5,459,030 also issued to Lin teaches a platelet storage medium containing 8-methoxypsoralen for use in a pathogen reduction process. U.S. Pat. Nos. 5,712,085, 5,908,742, 5,955,256, 5,965,349, 6,017,691 and 6,251,580 all disclose solutions for use in the pathogen reduction of blood which all include psoralen or psoralen derivatives as the photosensitizer. None of these disclosed solutions suggests the addition of glycolytic inhibitors to help in the pathogen reduction and subsequent storage of the pathogen reduced blood and/or blood products.

U.S. Pat. No. 5,120,649 issued Jun. 9, 1992 and related U.S. Pat. No. 5,232,844 issued Aug. 3, 1993 to Horowitz, et al., also disclose the need for the use of “quenchers” in combination with photosensitizers which attack lipid membranes, and U.S. Pat. No. 5,360,734 issued Nov. 1, 1994 to Chapman et al. also addresses this problem of prevention of damage to other blood components.

Photosensitizers which attack nucleic acids are known to the art. U.S. Pat. No. 5,342,752 issued Aug. 30, 1994 to Platz et al. discloses the use of compounds based on acridine dyes to reduce parasitic contamination in blood matter comprising red blood cells, platelets, and blood plasma protein fractions. These materials, although of fairly low toxicity, do have some toxicity e.g. to red blood cells. This patent fails to disclose an apparatus for decontaminating blood on a flow-through basis. U.S. Pat. No. 5,798,238 to Goodrich, Jr., et al., discloses the use of quinolone and quinolone compounds for inactivation of viral and bacterial contaminants.

Binding of DNA with photoactive agents has been exploited in processes to reduce lymphocytic populations in blood as taught in U.S. Pat. No. 4,612,007 issued Sep. 16, 1986 and related U.S. Pat. No. 4,683,889 issued Aug. 4, 1987 to Edelson.

The vitamin riboflavin (7,8-dimethyl-10-ribityl isoalloxazine) has also been reported to attack nucleic acids. U.S. Pat. Nos. 6,258,577 and 6,277,337 issued to Goodrich et al. disclose the use of riboflavin as an endogenous photosensitizer and light to inactivate microorganisms which may be contained in blood or blood products. U.S. Pat. No. 6,268,120 to Platz et al. discloses riboflavin derivatives which may be used to inactivate microorganisms.

All publications referred to herein are hereby incorporated by reference to the extent not inconsistent herewith.

The present invention is directed to a solution containing glycolysis inhibitors for irradiating and storing platelets to help maintain the quality of platelets during a pathogen reduction process, as well as for long term storage of the pathogen reduced platelet product.

### SUMMARY OF THE INVENTION

This invention relates to the addition of glycolysis inhibitors to solutions containing platelets which have been or which will be subjected to a pathogen reduction process, in order to help maintain the quality of the platelets during the process as well as afterwards during storage. More particularly, the invention relates to the addition of 2-deoxy-D-glucose to a platelet storage solution. In a further embodiment, 2-deoxy-D-glucose is added to a solution containing platelets in an amount between 1-10 mM.

### BRIEF DESCRIPTION OF THE FIGURES

FIG. 1 is a graph showing the production of lactate by pathogen reduced platelets as a function of time. FIG. 2 is a graph showing the consumption of glucose by pathogen reduced platelets as a function of time. FIG. 3 is a graph showing changes in pH by pathogen reduced platelets as a function of time. FIG. 4 shows the percentage of platelets which become activated over time after being subjected to a pathogen reduction procedure. FIG. 5 shows an embodiment of this invention using a bag to contain the fluid being treated with the photosensitizer and glycolytic inhibitor and a shaker table to agitate the fluid while exposing to photoradiation from a light source.

### DETAILED DESCRIPTION OF THE INVENTION

A solution is provided for stabilizing platelets either before, during or after a pathogen reduction process.

The photosensitizers useful in this invention include any photosensitizers known to the art to be useful for inactivating microorganisms or other infectious particles. A “photosensitizer” is defined as any compound which absorbs radiation of one or more defined wavelengths and subsequently utilizes the absorbed energy to carry out a chemical process. Examples of such photosensitizers include porphyrins, psoralens, dyes such as neutral red, methylene blue, acridine, toluidines, flavine (acriflavine hydrochloride) and phenothiazine derivatives, coumarins, quinolones, quinones, and anthroquinones. Photosensitizers of this invention may include compounds which preferentially adsorb to nucleic acids, thus focusing their photodynamic effect upon microorganisms and viruses with little or no effect upon accompanying cells or proteins. Other photosensitizers are also useful in this invention, such as those using singlet oxygen-dependent mechanisms.

Most preferred are endogenous photosensitizers. The term “endogenous” means naturally found in a human or mammalian body, either as a result of synthesis by the body or because of ingestion as an essential foodstuff (e.g. vitamins) or formation of metabolites and/or byproducts in vivo. Examples of such endogenous photosensitizers are alloxazines such as 7,8-dimethyl-10-ribityl isoalloxazine (riboflavin), 7,8,10-trimethylisoalloxazine (lumiflavin), 7,8-dimethylalloxazine (lumichrome), isoalloxazine-adenine dinucleotide (flavine adenine dinucleotide [FAD]), alloxazine mononucleotide (also known as flavine mononucleotide [FMN] and riboflavine-5-phosphate), vitamin Ks, vitamin L, their metabolites and precursors, and napththoquinones, naphthalenes, naphthols and their derivatives having planar molecular conformations. The term “alloxazine” includes isoalloxazines. Endogenously-based derivative photosensitizers include synthetically derived analogs and homologs of endogenous photosensitizers which may have or lack lower (1-5) alkyl or halogen substituents of the photosensitizers from which they are derived, and which preserve the function and substantial non-toxicity thereof. When endogenous photosensitizers are used, particularly when such photosensitizers are not inherently toxic or do not yield toxic photoproducts after photoradiation, no removal or purification step is required after decontamination, and the treated product can be directly administered to a patient by any methods known in the art. Preferred endogenous photosensitizers are:

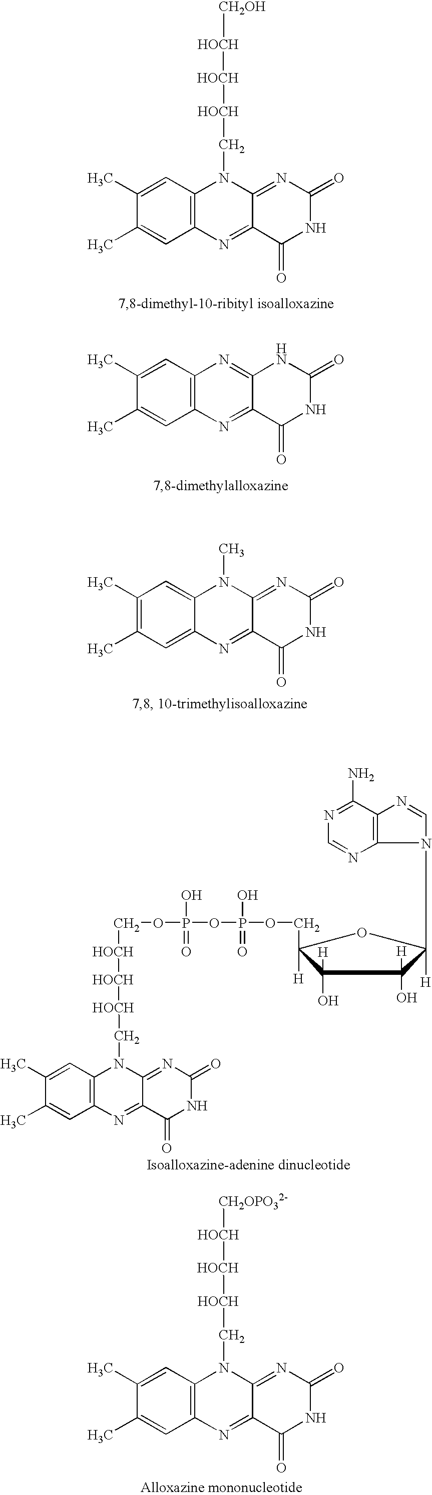

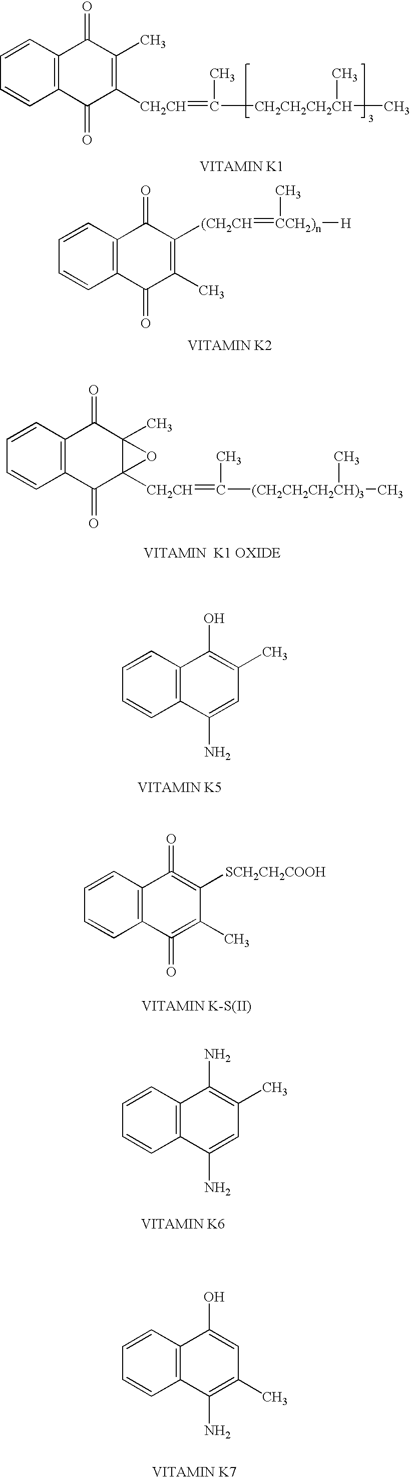

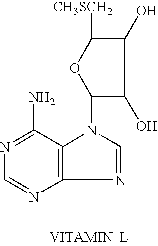

The method of this invention requires mixing the photosensitizer and the glycolysis inhibitor with the fluid containing platelets to be pathogen reduced. Mixing may be done by simply adding the photosensitizer or a solution containing the photosensitizer to a fluid to be pathogen reduced. Similarly, the glycolysis inhibitor or a solution containing the glycolysis inhibitor may be added to the platelets either before the pathogen reduction procedure or after the procedure. In one embodiment, the material to be decontaminated to which a photosensitizer and glycolysis inhibitor has been added is flowed past a photoradiation source, and the flow of the material generally provides sufficient turbulence to distribute the photosensitizer and glycolsis inhibitor throughout the fluid to be pathogen reduced. A mixing step may optionally be added. In another embodiment, the fluid, photosensitizer and glycolysis inhibitor are placed in a photopermeable container and irradiated in batch mode, preferably while agitating the container to fully distribute the photosensitizer and expose all the fluid to the radiation.

The amount of photosensitizer to be mixed with the fluid will be an amount sufficient to adequately inactivate the reproductive ability of a pathogen. As taught herein, optimal concentrations for desired photosensitizers may be readily determined by those skilled in the art without undue experimentation. Preferably the photosensitizer is used in a concentration of at least about 1 μM up to the solubility of the photosensitizer in the fluid. For 7,8-dimethyl-10-ribityl isoalloxazine a concentration range between about 1 μM and about 160 μM is preferred, preferably about 50 μM.

The fluid containing the photosensitizer and glycolytic inhibitor is exposed to photoradiation of the appropriate wavelength to activate the photosensitizer, using an amount of photoradiation sufficient to activate the photosensitizer as described above, but less than that which would cause severe damage to the platelets being pathogen reduced. The addition of glycolysis inhibitor as described may help platelets maintain their viability after exposure to a photosensitizer and light.

The wavelength used will depend on the photosensitizer selected, as is known to the art or readily determinable without undue experimentation following the teachings hereof. Preferably the light source is a fluorescent or luminescent source providing light of about 300 nm to about 700 nm, and more preferably about 308 nm to about 447 m of radiation. Wavelengths in the ultraviolet to visible range are useful in this invention. The light source or sources may provide light in the visible range, light in the ultraviolet range, or may be a mixture of light in the visible and ultraviolet ranges.

The activated photosensitizer is capable of inactivating the infectious particles present, such as by interfering to prevent their replication. Specificity of action of the photosensitizer is conferred by the close proximity of the photosensitizer to the nucleic acid of the particle and this may result from binding of the photosensitizer to the nucleic acid. “Nucleic acid” includes ribonucleic acid (RNA) and deoxyribonucleic acid (DNA). Other photosensitizers may act by binding to cell membranes or by other mechanisms. The photosensitizer may also be targeted to the particles to be inactivated by covalently coupling to an antibody, preferably a specific monoclonal antibody to the particle.

The fluid containing the photosensitizer and glycolysis inhibitor may be flowed into a photopermeable container for irradiation. The term “container” refers to a closed or open space, which may be made of rigid or flexible material, e.g., may be a bag or box or trough. It may be closed or open at the top and may have openings at both ends, e.g., may be a tube or tubing, to allow for flow-through of fluid therein. A cuvette has been used to exemplify one embodiment of the invention involving a flow-through system. Collection bags, such as those used with the Trima® and Spectra™ apheresis systems manufactured by Gambro BCT, Inc. (Lakewood, Colo., USA), have been used to exemplify another embodiment involving batch-wise treatment of the fluid.

The term “photopermeable” means the material of the container is adequately transparent to photoradiation of the proper wavelength for activating the photosensitizer. In the flow-through system, the container has a depth (dimension measured in the direction of the radiation from the photoradiation source) sufficient to allow photoradiation to adequately penetrate the container to contact photosensitizer molecules at all distances from the light source and ensure inactivation of infectious particles in the fluid to be decontaminated, and a length (dimension in the direction of fluid flow) sufficient to ensure a sufficient exposure time of the fluid to the photoradiation. The materials for making such containers, depths and lengths of containers may be easily determined by those skilled in the art without undue experimentation following the teachings hereof, and together with the flow rate of fluid through the container, the intensity of the photoradiation and the absorptivities of the fluid components, will determine the amount of time the fluid needs to be exposed to photoradiation. For 7,8-dimethyl-10-ribityl isoalloxazine, a preferred amount of radiation is between about 1 J/cm² to 120 J/cm².

In another embodiment involving batch-wise treatment, the fluid to be treated is placed in a photopermeable container which is agitated and exposed to photoradiation for a time sufficient to substantially inactivate the pathogens which may be present, but not enough to destroy the viability of the blood or blood component. The photopermeable container is preferably a blood bag made of transparent or semitransparent plastic, and the agitating means is preferably a shaker table. The photosensitizer and/or glycolysis inhibitor may be added to the container in powdered or liquid form and the container agitated to mix the photosensitizer with the fluid and to adequately expose all the fluid to the photoradiation to ensure inactivation of the particles.

Photosensitizer and/or glycolysis inhibitor may be added to or flowed into the photopermeable container containing the blood components to be inactivated or pathogen reduced. In one embodiment, the photosensitizer and/or glycolysis inhibitor is added to the fluid which is used to suspend the blood components to be pathogen inactivated. In another embodiment, the photosensitizer and/or glycolysis inhibitor may be added to the blood components to be inactivated and the suspension fluid.

In the description above it is further understood that the photosensitizer and glycolysis inhibitor can be each added separately.

Quenchers may also be added to the fluid to make the process more efficient and selective. Such quenchers include antioxidants or other agents to prevent damage to desired fluid components or to improve the rate of inactivation of pathogens and are exemplified by adenine, histidine, cysteine, tyrosine, tryptophan, ascorbate, N-acetyl-L-cysteine, propyl gallate, glutathione, mercaptopropionylglycine, dithiothreotol, nicotinamide, BHT, BHA, lysine, serine, methionine, glucose, mannitol, trolox, glycerol, and mixtures thereof.

In decontamination systems of this invention, the photoradiation source may be connected to the photopermeable container for the fluid by means of a light guide such as a light channel or fiber optic tube which prevents scattering of the light between the source and the container for the fluid, and more importantly, prevents substantial heating of the fluid within the container. Direct exposure to the light source may raise temperatures as much as 10 to 15° C., especially when the amount of fluid exposed to the light is small, which can cause denaturization of blood components. Use of the light guide keeps any heating to less than about 2° C. The method may also include the use of temperature sensors and cooling mechanisms where necessary to keep the temperature below temperatures at which desired proteins in the fluid are damaged. Preferably, the temperature is kept between about 0° C. and about 45° C., more preferably between about 4° C. and about 37° C., and most preferably about 22° C.

The photoradiation source may be a simple lamp or may consist of multiple lamps radiating at differing wavelengths. The photoradiation source should be capable of delivering from about 1 J/cm² to at least about 120 J/cm².

Any means for adding the photosensitizer and glycolysis inhibitor to the fluid to be decontaminated and for placing the fluid in the photopermeable container known to the art may be used, such means typically including flow conduits, ports, reservoirs, valves, and the like.

For endogenous photosensitizers and derivatives having sugar moieties, the pH of the solution is preferably kept low enough, as is known to the art, to prevent detachment of the sugar moiety. Preferably the photosensitizer is added to the fluid to be decontaminated in a pre-mixed aqueous solution, e.g., in water, storage buffer or suspension solution.

The photopermeable container for the flow-through system may be a transparent cuvette made of polycarbonate, glass, quartz, polystyrene, polyvinyl chloride, polyolefin, or other transparent material. The cuvette may be enclosed in a radiation chamber having photoreflective walls. A photoradiation enhancer such as a second photoradiation source or reflective surface may be placed adjacent to the cuvette to increase the amount of photoradiation contacting the fluid within the cuvette. The system preferably includes a pump for adjusting the flow rate of the fluid to desired levels to ensure substantial decontamination as described above. The cuvette has a length, coordinated with the flow rate therethrough, sufficient to expose fluid therein to sufficient photoradiation to effect substantial decontamination thereof.

Also preferably the cuvette is spaced apart from the light source a sufficient distance that heating of the fluid in the cuvette does not occur, and light is transmitted from the light source to the cuvette by means of a light guide.

Decontamination systems as described above may be designed as stand-alone units or may be easily incorporated into existing apparatuses known to the art for reducing pathogens in blood or blood components. The process is further described and is incorporated in its entirety to the amount not inconsistent in U.S. Pat. Nos. 6,277,337 and 6,258,577.

FIG. 5 depicts an embodiment of this invention in which fluid to be decontaminated is placed in a bag 284 equipped with an inlet port 282, through which photosensitizer and glycolysis inhibitor 290 may be added from flask 286 via pour spout 288. Shaker table 280 is activated to agitate the bag 284 to mix the fluid to be decontaminated, the photosensitizer and the glycolysis inhibitors together while photoradiation source 260 is activated to irradiate the fluid and photosensitizer in bag 284. Alternatively, the bag can be provided prepackaged to contain photosensitizer and glycolysis inhibitors and the fluid is thereafter added to the bag.

It has been observed that one possible side effect of a pathogen reduction process is that when platelets are subjected to UV light, the mitochondria of the platelets have a greater chance of suffering at least some damage. If mitochondrial function is suppressed by UV light, platelets are unable to create ATP (energy) through aerobic respiration. If platelets are unable to create energy through aerobic respiration, they will create energy through the glycolysis pathway. As described above, one metabolite produced by the glycolysis pathway is lactic acid. Lactic acid buildup within cells causes the pH of the solution to drop. Such a drop in pH causes decreased cell quality during storage.

One way to prevent this pH drop and subsequent drop in cell quality would be to prevent the buildup of lactic acid. This may be done by using an agent or agents which block or slow glycolysis.

2-deoxy-D-glucose is one such agent which slows the rate of glycolysis by inhibiting enzymatic processes within the glycolytic chain and may be added to the fluid to be pathogen reduced in the manner as described above.

In one embodiment, 2-deoxy-D-glucose is added to a fluid containing a platelet suspension before the platelets are subjected to a pathogen reduction procedure. In a preferred embodiment, 2-deoxy-D-glucose is added to the fluid at a concentration of 1 mM to 10 mM. The presence of this agent should slow the production of lactic acid during storage, allowing maintenance of pH, and consequently, cell quality during prolonged storage should be better maintained. The platelets could then be pathogen reduced according the pathogen reduction procedure described above.

In an alternative embodiment, 2-deoxy-D-glucose may be added to platelets after a pathogen reduction procedure, to aid in storage of the pathogen reduced platelets. The following aqueous blood component additive solutions are some examples of readily available commercial platelet additive solutions which may be used with the present invention.

### EXAMPLE 1

This example compares solvents which are novel blood component additive solutions for addition to platelets separated from whole blood. Six commercially available solutions were used: PAS II, PSMI-pH, PlasmaLyte A, SetoSol, PAS m, and PAS. To each known solution was added an effective amount of an endogenous photosensitizer, 7,8-dimethyl-10-ribityl isoalloxazine and 2-deoxy-D-glucose. The photosensitizer may be present in the various solutions at any desired concentration from about 1 μM up to the solubility of the photosensitizer in the fluid, or dry medium, and preferably about 10 μM. For 7,8-dimethyl-10-ribityl isoalloxazine a concentration range between about 1 μM and about 160 μM is preferred, preferably about 50 μM. The composition of each solution is shown in Table 1a below, and varies in the amount of blood component additives present. The blood additive components may be in a physiological solution, as well as a dry medium adapted to be mixed with a solvent, including tablet, pill or capsule form.

TABLE 1a

| Blood Component | Platelet Storage Solution | | | | | |
|---|---|---|---|---|---|---|
| Additive | PSS 1 | PSS 2 | PSS 3 | PSS 4 | PSS 5 | P55 6 |
| KCl (mM) | | 5.0 | 5.0 | 5.0 | 5.1 | |
| CaCl₂ (m/M) | | | | | 1.7 | |
| MgCl₂ (m/M) | | | 3.0 | 3.0 | | |
| MgSO₄ (mM) | | | | | 0.8 | |
| sodium citrate (mM) | 10.0 | 23.0 | 23.0 | 17.0 | 15.2 | 12.3 |
| citric acid (mM) | | | | | 2.7 | |
| NaHCO₃ (mM) | | | | | 35.0 | |
| Na₂HPO₄ (mM) | | 25.0 | | 25.0 | 2.1 | 28.0 |
| sodium acetate (mM) | 30.0 | | 27.0 | 23.0 | | 42.0 |
| sodium gluconate (mM) | | | 23.0 | | | |
| glucose (mM) | | | | 23.5 | 38.5 | |
| maltose (mM) | | | | 28.8 | | |
| 7,8-dimethyl 10-ribityl isoalloxazine | 50.0 | 50.0 | 50.0 | 50.0 | 50.0 | 50.0 |
| (μM) | | | | | | |
| 2-deoxy-D-glucose (μM) | 10.0 | 10.0 | 10.0 | 10.0 | 10.0 | 10.0 |

In Example 1, the platelet storage solution PSS 1 comprises a physiological saline solution, tri-sodium citrate at a concentration of approximately about 10 mM, sodium acetate at a concentration of approximately about 30 mM, 7, 8-dimethyl-10-ribityl isoalloxazine at a concentration of about 50 μM and 2-deoxy-D-glucose at a concentration of approximately 10 μM.

In Example 1, the platelet storage solution PSS 2 comprises a physiological saline solution, potassium chloride at a concentration of approximately about 5 mM, tri-sodium citrate at a concentration of approximately about 23 mM, a mixture of monosodium phosphate and dibasic sodium phosphate at a concentration of approximately about-25 mM, 7, 8-dimethyl-10-ribityl isoalloxazine at a concentration of about 50 μM and 2-deoxy-D-glucose at a concentration of approximately 10 μM.

The platelet storage solution PSS 3 comprises a physiological saline solution, potassium chloride at a concentration of approximately about 5 mM, magnesium chloride at a concentration of approximately about 3 mM, tri-sodium citrate at a concentration of approximately about 23 mM, sodium acetate at a concentration of approximately about 27 mM, sodium gluconate at a concentration of approximately about 23 mM, 7, 8-dimethyl-10-ribityl isoalloxazine at a concentration of about 50 μM and 2-deoxy-D-glucose at a concentration of approximately 10 μM.

The platelet storage solution PSS 4 comprises a physiological saline solution, potassium chloride at a concentration of approximately about 5 mM, magnesium chloride at a concentration of approximately about 3 mM, tri-sodium citrate at a concentration of approximately about 17 mM, sodium phosphate at a concentration of approximately about 25 mM, sodium acetate at a concentration of approximately about 23 mM, glucose at a concentration of approximately about 23.5 mM, maltose at a concentration of approximately about 28.8 mM, 7, 8-dimethyl-10-ribityl isoalloxazine at a concentration of about 50 μM and 2-deoxy-D-glucose at a concentration of approximately 10 μM.

Platelet storage solution PSS 5 comprises a physiological saline solution, potassium chloride at a concentration of approximately about 5.1 mM, calcium chloride at a concentration of approximately about 1.7 mM, magnesium sulfate at a concentration of approximately about 0.8 mM, tri-sodium citrate at a concentration of approximately about 15.2 mM, citric acid at a concentration of approximately about 2.7 mM, sodium bicarbonate at a concentration of approximately about 35 mM, sodium phosphate at a concentration of approximately about 2.1 mM, glucose at a concentration of approximately about 38.5 mM, 7,8-dimethyl-10-ribityl isoalloxazine at a concentration of about 10 μM and 2-deoxy-D-glucose at a concentration of approximately 10 μM.

In Example 1, the platelet storage solution PSS 6 comprises a physiological saline solution, tri-sodium citrate at a concentration of approximately about 12.3 mM, sodium phosphate at a concentration of approximately about 28 mM, sodium acetate at a concentration of approximately about 42 mM, 7,8-dimethyl-10-ribityl isoalloxazine at a concentration of about 50 μM and 2-deoxy-D-glucose at a concentration of approximately 10 μM.

The physiologic saline solution may be replaced with a solvent comprising water and an effective amount of sodium chloride. 2-deoxy-D-glucose may also be added to a solution containing saline or water and an effective amount of 7,8-dimethyl-10-ribityl isoalloxazine.

The blood additive solution could comprise other additive solutions including an effective amount of 7, 8-dimethyl-10-ribityl isoalloxazine and an inhibitor of glycolysis such as 2-deoxy-D-glucose in a liquid, pill or dry medium form. PSS 7, PSS 8 and PSS 9 are further examples of such blood additive solutions set forth in Table 1b below. A quencher such as any disclosed above may also be added.

TABLE 1b

| | Platelet Storage Solution | | |
|---|---|---|---|
| Blood Component Additive | PSS 7 | PSS 8 | P55 9 |
| NaCl (mM) | 115.0 | 78.3 | 68.5 |
| potassium cloride (mM) | | 5.7 | 5.0 |
| MgCl₂ (mM) | | 1.7 | 1.5 |
| sodium citrate (mM) | 10.0 | | |
| sodium phosphate (monobasic) | 6.2 | 5.4 | 8.5 |
| sodium phosphate (dibasic) | 19.8 | 24.6 | 21.5 |
| sodium acetate (mM) | 30.0 | 34.3 | 30.0 |
| 7,8-dimethyl 10-ribityl isoalloxazine (μM) | 50.0 | variable | 50.0 |
| 2-deoxy-D-glucose (μM) | 10.0 | 10.0 | 10.0 |

As described in Table 1b, PSS 7 was prepared in RODI water and sodium chloride at a concentration of approximately 115 mM, sodium citrate at a concentration of approximately 10.0 mM, sodium phosphate (monobasic) at a concentration of approximately 6.2 mM, sodium phosphate (dibasic) at a concentration of approximately 19.8 mM, sodium acetate at a concentration of approximately 30.0 mM, 7,8-dimethyl 10-ribityl isoalloxazine at a concentration of approximately 50.0 μM and 2-deoxy-D-glucose at a concentration of approximately 10 μM. It has a pH of 7.2.

PSS 8 was prepared in RODI water and comprises and sodium chloride at a concentration of approximately 78.3 mM, potassium chloride at a concentration of approximately 5.7 mM, magnesium chloride at a concentration of approximately 1.7 mM, sodium phosphate (monobasic) at a concentration of approximately 5.4 mM, sodium phosphate (dibasic) at a concentration of approximately 24.6 MM, sodium acetate at a concentration of approximately 34.3 mM, a variable concentration of 7,8-dimethyl 10-ribityl isoalloxazine and 2-deoxy-D-glucose at a concentration of approximately 10 μM. It has a pH of 7.4, and an osmolarity of 297 mmol/kg.

PSS 9 was prepared in RODI water and comprises and sodium chloride at a concentration of approximately 68.5 mM, potassium chloride at a concentration of approximately 5.0 mM, magnesium chloride at a concentration of approximately 1.5 mM, sodium phosphate (monobasic) at a concentration of approximately 8.5 mM, sodium phosphate (dibasic) at a concentration of approximately 21.5 mM, sodium acetate at a concentration of approximately 30.0 mM, 7,8-dimethyl 10-ribityl isoalloxazine at a concentration of approximately 50.0 μM and 2-deoxy-D-glucose at a concentration of approximately 10 μM. It has a pH of 7.2, and an osmolarity of 305 mmol/kg.

It is understood that in PSS 7, PSS 8 and PSS 9 the RODI water and sodium chloride can be replaced with a saline solution.

It is also contemplated that a platelet additive solution in accordance with this invention can comprise 7, 8-dimethyl-10-ribityl isolloxazine, 2-deoxy-D-glucose and an quencher as described above.

### EXAMPLE 2

The quality of platelets after a pathogen reduction procedure may be measured using standard measures of platelet quality known in the art.

The following figures graph the quality of pathogen reduced platelets over a five day storage period. Platelets were separated from whole blood and collected using a blood collection device such as the COBE Spectra™ or TRIMA® apheresis systems available from Gambro BCT Inc., Lakewood, Colo., USA. However, it should be noted that any device known in the art for separating blood into components may be used to collect platelets without departing from the spirit and scope of the present invention.

Collected platelets were suspended in a volume of 278 mL of fluid containing 50 μL riboflavin and either with or without a glycolytic inhibitor. 2-deoxy-D-glucose is one example of a glycolytic inhibitor which may be used in the present invention. The platelets were irradiated in a Sengewald bag (available from Sengewald Verpackungen GmbH & Co. KG) (however any bag known in the art may be used) at 7 J/cm² with a 320 nm broad band eximer light.

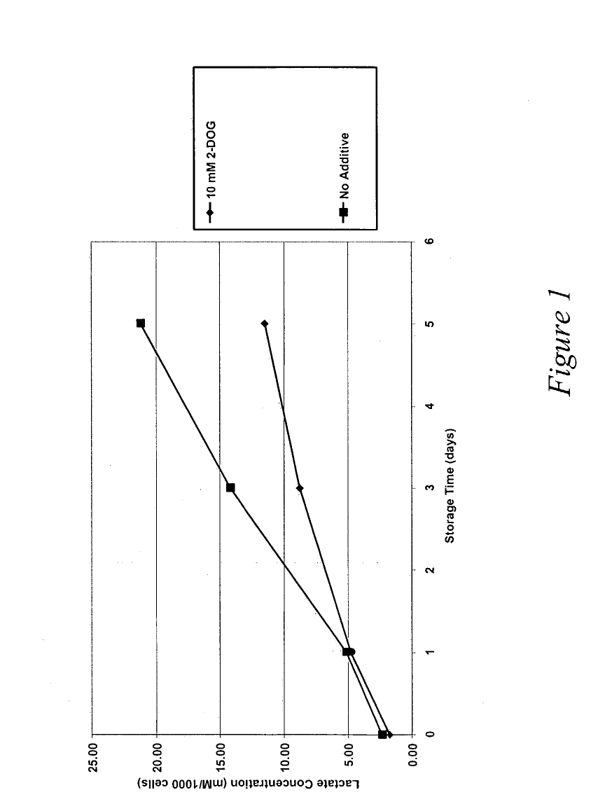

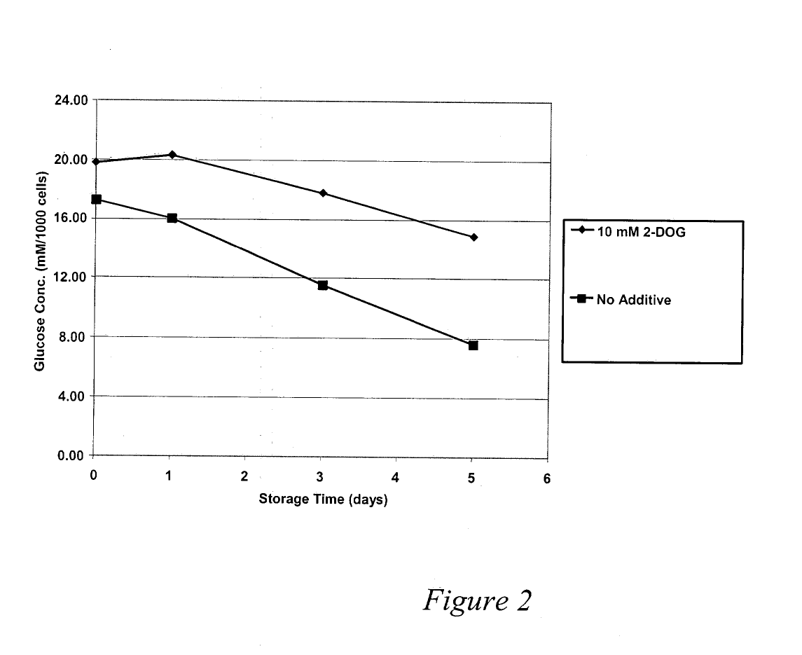

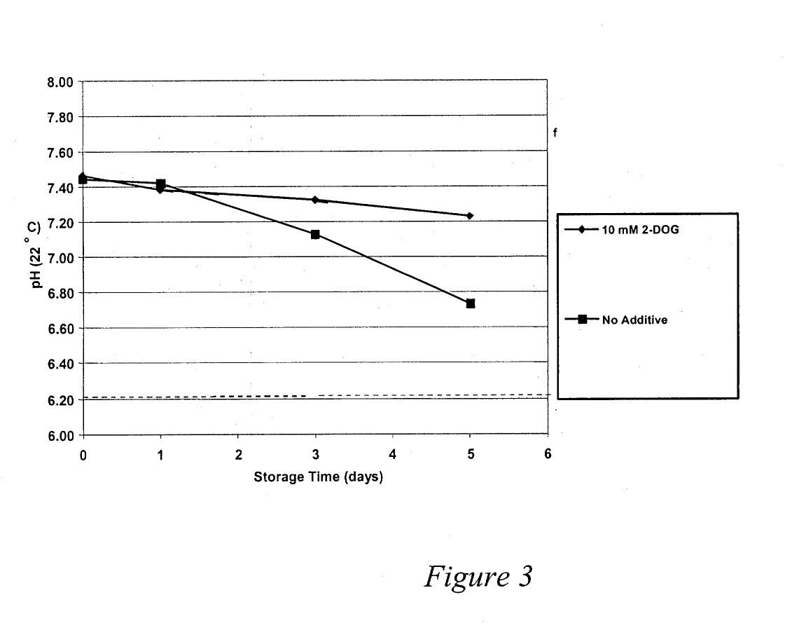

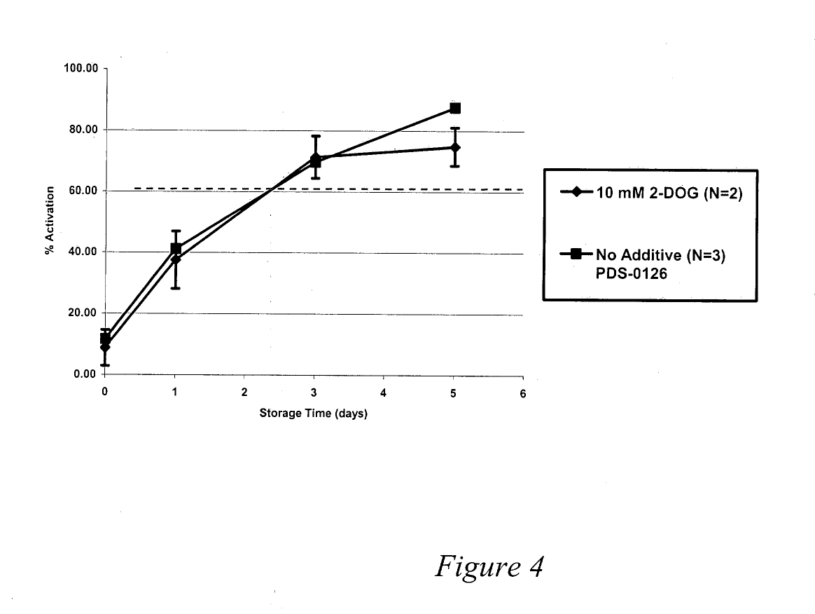

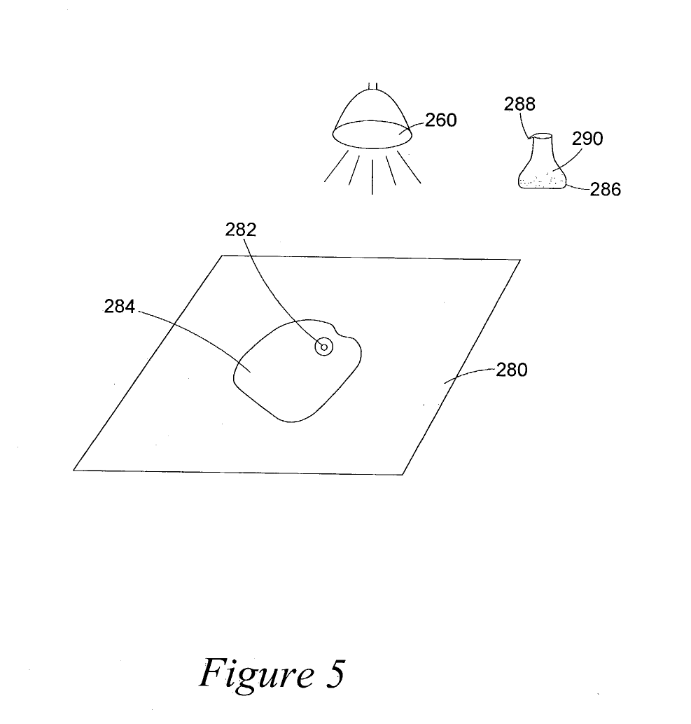

FIG. 1 shows lactate production by pathogen reduced platelets over a five day storage period. The platelets which were pathogen reduced and stored in a solution containing 10 mM 2-deoxy-D-glucose show a marked decrease in the production of lactic acid over five days in storage as compared to platelets which were pathogen reduced and stored in solution without 2-deoxy-D-glucose. This is an expected result since 2-deoxy-D-glucose blocks or slows the glycolysis pathway and consequently the breakdown of pyruvate into lactate would be significantly decreased.

FIG. 2 shows glucose consumption of pathogen reduced platelets over time. As can be seen from FIG. 2, glucose is consumed at a slower rate by pathogen reduced platelets in a solution containing 10 mM 2-deoxy-D-glucose than by platelets containing no 2-deoxy-D-glucose. This result is expected because 2-deoxy-D-glucose inhibits or slows down the glycolytic pathway. By inhibiting or slowing down the glycolysis pathway, platelets are forced to produce ATP through the citric acid pathway, and therefore are able to consume less glucose then platelets which do not have blocked or slowed glycolysis pathways.

FIG. 3 shows the drop in pH over time of a solution of pathogen reduced platelets. As shown, 10 mM 2-deoxy-D-glucose appears to maintain the pH of the platelets at approximately pH 7.40. Platelets without 2-deoxy-D-glucose experience a rapid drop in pH. Such results are expected because lactic acid is not produced at high rates if the glycolysis pathway is inhibited, and consequently, the pH of the solution remains at a relatively constant level.

FIG. 4 is a graph showing GMP-140 expression on the surface of pathogen treated platelets over a five day storage period. GMP-140 is a marker which appears on the surface of platelets which are activated. When transfused into a recipient, platelets which are activated during storage have a higher percentage of being quickly removed from the recipient's circulatory system than non-activated platelets. As shown in FIG. 4, platelets which were pathogen reduced and stored in solutions containing 2-deoxy-D-glucose have a lower percentage of GMP-140 expression than platelets which were pathogen reduced and stored in solutions which do not have glycolytic inhibitors.

In addition to 2-deoxy-D-glucose, other sugars may find similar utility as glycolytic inhibitors. These include xylose, ribose, arabinose, and lyxose. The fact that all of these agents are natural, non-toxic and well tolerated in vivo make them ideal candidates for use in pathogen reduction and storage solutions. The above mentioned glycolytic inhibitors may be used either alone or in combination.

It will be readily understood by those skilled in the art that the foregoing description has been for purposes of illustration only and that a number of changes may be made without departing from the scope of the invention. For example, photosensitizers other then those mentioned may be used, preferably photosensitizers which bind to nucleic acid and thereby keep it from replicating, and more preferably those which are not toxic and do not have toxic breakdown products. In addition, equivalent structures to those described herein for inhibiting the glycolytic pathway in platelets may be readily devised without undue experimentation by those skilled in the art following the teachings hereof.

1. A pathogen reduction solution for suspending platelets undergoing a pathogen reduction procedure comprising;
an endogenous photosensitizer; and
a glycolytic inhibitor.

2. A pathogen reduction solution of claim 1 further comprising a solvent.

3. The pathogen reduction solution of claim 1 wherein the endogenous photosensitizer further comprises 7,8-dimethyl-10-ribityl isoalloxazine.

4. The pathogen reduction solution of claim 1 wherein the glycolytic inhibitor is selected from the group consisting of 2-deoxy-D-glucose, xylose, arabinose and lyxose.

5. The pathogen reduction solution of claim 1 wherein the glycolytic inhibitor is added to the solution at a concentration of between 1 mM to 10 mM.

6. The pathogen reduction solution of claim 1 wherein the glycolytic inhibitor is 2-deoxy-D-glucose at a concentration of 10 mM.

7. The pathogen reduction solution of claim 2 wherein the solvent is selected from the group consisting of PSS 1, PSS 2, PSS 3, PSS 4, PSS 5, PSS 6, PSS 7, PSS 8 and PSS 9.

8. The pathogen reduction solution of claim 2 wherein the solvent is selected from the group consisting of saline and water.

9. The pathogen reduction solution of claim 1 further comprising a quencher selected from the group consisting of adenine, histidine, cysteine, tyrosine, tryptophan, ascorbate, N-acetyl-L-cysteine, propyl gallate, glutathione, mercaptopropionylglycine, dithiothreotol, nicotinamide, BHT, BHA, lysine, serine, methionine, glucose, mannitol, trolox, glycerol, and mixtures thereof.

10. A storage solution for suspending platelets which have undergone a pathogen reduction procedure comprising;
an endogenous photosensitizer; and
a glycolytic inhibitor.

11. A storage solution of claim 10 further comprising a solvent.

12. The storage solution of claim 10 wherein the endogenous photosensitizer further comprises 7, 8-dimethyl-10-ribityl isoalloxazine.

13. The storage solution of claim 10 wherein the glycolytic inhibitor is selected from the group consisting of 2-deoxy-D-glucose, xylose, arabinose and lyxose.

14. The pathogen reduction solution of claim 10 wherein the glycolytic inhibitor is added to the solution at a concentration of between 1 mM to 10 mM.

15. The pathogen reduction solution of claim 10 wherein the glycolytic inhibitor is 2-deoxy-D-glucose at a concentration of 10 mM.

16. The storage solution of claim 11 wherein the solvent is selected from the group consisting of PSS 1, PSS 2, PSS 3, PSS 4, PSS 5, PSS 6, PSS 7, PSS 8 and PSS 9.

17. The storage solution of claim 11 wherein the solvent is selected from the group consisting of saline and water.

18. The pathogen reduction solution of claim 10 further comprising a quencher selected from the group consisting of adenine, histidine, cysteine, tyrosine, tryptophan, ascorbate, N-acetyl-L-cysteine, propyl gallate, glutathione, mercaptopropionylglycine, dithiothreotol, nicotinamide, BHT, BHA, lysine, serine, methionine, glucose, mannitol, trolox, glycerol, and mixtures thereof.

19. A method of substantially reducing pathogens in a fluid containing platelets comprising:
adding an effective, non-toxic amount of an endogenous photosensitizer to the fluid;
adding an effective amount of a glycolytic inhibitor; and
exposing the fluid to photoradiation sufficient to activate the photosensitizer and substantially reduce the pathogens.

20. The method of claim 19 wherein the endogenous photosensitizer further comprises 7,8-dimethyl-10-ribityl isoalloxazine.

21. The method of claim 19 wherein the glycolytic inhibitor is selected from the group consisting of 2-deoxy-D-glucose, xylose, arabinose and lyxose.

22. The method of claim 19 wherein the glycolytic inhibitor is added to the solution at a concentration of between 1 mM to 10 mM.

23. The method of claim 19 wherein the glycolytic inhibitor is 2-deoxy-D-glucose at a concentration of 10 mM.

24. A method of maintaining the viability of platelets during a pathogen reduction procedure comprising:
adding an effective, non-toxic amount of an endogenous photosensitizer to the fluid;
adding an effective amount of a glycolytic inhibitor; and
exposing the fluid to photoradiation sufficient to activate the photosensitizer and substantially reduce the pathogens.

25. A method of maintaining the viability of platelets after a pathogen reduction procedure comprising:
adding an effective, non-toxic amount of an endogenous photosensitizer to the fluid;
exposing the fluid to photoradiation sufficient to activate the photosensitizer and substantially reduce the pathogens and
adding an effective amount of a glycolytic inhibitor.
